(19) United States
(12) Patent Application Publication
(10) Pub. No.: US 2026/0308458 A1
(43) Pub. Date: Oct. 8, 2026

(54) DIRECT HEATING ELECTRIC TUBE FOR UPGRADING HYDROCARBONS WITH CONTROLLED TUBE MATERIAL TEMPERATURE

(71) Applicant: Dow Global Technologies LLC, Midland, MI (US)

(72) Inventors: Quan Yuan, Sugarland, TX (US); Huan T. Ngo, Sherwood Park (CA); Georgios Bellos, Terneuzen (NL); Deepak Kumar, Sugarland, TX (US); Albert D. Harvey, III, Conroe, TX (US); Lihui Wang, Lake Jackson, TX (US); David Burns, Huntsville, TX (US)

(73) Assignee: Dow Global Technologies LLC, Midland, MI (US)

(21) Appl. No.: 19/483,319

(22) PCT Filed: May 17, 2023
(86) PCT No.: PCT/GR2023/000020

§ 371 Date: Nov. 11, 2025

(51) Int. Cl. C10G 9/24; C07C 4/04; C10G 9/20

(52) U.S. Cl. CPC C10G 9/24; C07C 4/04; C10G 9/20; C10G 2400/20

(57) ABSTRACT

A heating apparatus for upgrading a hydrocarbon fluid comprises a current voltage source and has at least one electrically conductive tube, wherein the tube material temperature is controlled with geometries that alter the resistance along the U-shaped portion of the electrically conductive tube such that the resistance is less than the resistance along the straight portions of the electrically conductive tube. The heating apparatus can be used for steam cracking process to produce ethylene.

### FIELD

Embodiments of the present disclosure generally relate to systems by which hydrocarbons are upgraded.

### BACKGROUND

Steam cracking processes to produce ethylene conventionally involve the combustion of fossil fuels to provide process heat. This results in substantial greenhouse gas emission. Steam cracking processes to produce ethylene conventionally involve the combustion of fossil fuels to provide process heat. This results in substantial greenhouse gas emission. Steam cracking accounted for 200 Mt of emitted CO₂ in 2000, which represents a significant fraction of the global anthropogenic emissions of 33.4 gigatonnes CO₂ per year in 2019. The electrification of steam cracking substantially reduces the environmental impact of the process by reducing CO₂ emissions.

### SUMMARY

In the application of direct electrical heating to large scale steam cracking, coil U-bends are typically deployed on cracker coils to increase reaction time by increasing reactor length without increasing overall process footprint. Longer reactor tubes also provide more surface area for a given amount of heat input, which can reduce the tube material temperature (TMT). Therefore, coil U-bends are an important component of the direct electrically heated reactor tube system. The electrically generated heat profile delivered to the process tubes affects the process yield and TMT. Excessive tube metal temperature leads to more rapid coke formation rate, accelerated rates of carburization, corrosion of coils, and imposes challenges on coil mechanical integrity. Low tube metal temperatures lead to poor conversion and reaction inefficiency. A key challenge is how to control the TMT within a range that provides an acceptably long coil lifetime that also provides advantages over coil lifetimes experienced in combustion-fired radiant furnaces.

Embodiments of this disclosure include an apparatus for upgrading a hydrocarbon fluid comprising a current voltage source and at least one electrically conductive tube comprising a hydrocarbon channel having a centerline. The at least one electrically conductive tube comprises at least one curve such that the electrically conductive tube comprises a plurality of straight portions and at least one U-shaped portion. The U-shaped portion comprises: an outer wall extending between an outer semicircle of the U-shaped portion and the hydrocarbon channel and having a thickness, w_{Uo}; an inner wall extending between an inner semicircle of the U-shaped portion and the hydrocarbon channel and having a thickness, w_{Ui}, wherein a ratio of w_{Uo} to w_{Ui} is within greater than or equal to 1.2 and less than or equal to

$$2 \times {( \frac{D + d}{D - d} )}^{2} ,$$

where D is a distance between centerlines of the plurality of straight portions and d is a tube diameter, wherein the current voltage source is electrically connected to the electrically conductive tube.

These and other embodiments are described in more detail in the Detailed Description.

### BRIEF DESCRIPTION OF FIGURES

The following detailed description of specific embodiments of the present disclosure can be best understood when read in conjunction with the following drawings, in which:

FIG. 1 is a schematic view of a process and apparatus for upgrading hydrocarbons, according to one or more of the present embodiments;

FIG. 2 is a side view of an apparatus for upgrading hydrocarbons, according to one or more of the present embodiments;

FIG. 3A is a schematic view of an apparatus for upgrading hydrocarbons;

FIG. 3B is a computational fluid dynamics (CFD) model of the TMT within a U-bend;

FIG. 4A is a schematic view of an apparatus for upgrading hydrocarbons;

FIG. 4B is a schematic view of an apparatus for upgrading hydrocarbons;

FIG. 4C is a schematic view of an electric circuit;

FIG. 4D is a schematic view of an apparatus for upgrading hydrocarbons, according to one or more of the present embodiments;

FIG. 4E is a graphical depiction of the relationship between the dimensions of an apparatus for upgrading hydrocarbons and resistance ratio, according to one or more of the present embodiments;

FIG. 5A is a CFD model of the process velocity magnitude within the U-bend of an apparatus for upgrading hydrocarbons, according to one or more of the present embodiments;

FIG. 5B is a CFD model of the power intensity within the U-bend of an apparatus for upgrading hydrocarbons, according to one or more of the present embodiments;

FIG. 6A is a front view of an apparatus for upgrading hydrocarbons, according to one or more of the present embodiments;

FIG. 6B is a top view of an apparatus for upgrading hydrocarbons, according to one or more of the present embodiments;

FIG. 7 is a front view of an apparatus for upgrading hydrocarbons, according to one or

more of the present embodiments;

FIG. 8 is a front view of an apparatus for upgrading hydrocarbons, according to one or more of the present embodiments;

FIG. 9A is a CFD model of the TMT within the U-bend, according to the present embodiments;

FIG. 9B is a CFD model of the TMT within the U-bend, according to the present embodiments; and

FIG. 10 is a graph of the temperature range of the TMT, according to one or more of the present embodiments.

### DETAILED DESCRIPTION

Embodiments of this disclosure include an apparatus for upgrading a hydrocarbon fluid comprising a current voltage source and at least one electrically conductive tube comprising a hydrocarbon channel having a centerline. The at least one electrically conductive tube comprises at least one curve such that the electrically conductive tube comprises a plurality of straight portions and at least one U-shaped portion. The U-shaped portion comprises: an outer wall extending between an outer semicircle of the U-shaped portion and the hydrocarbon channel and having a thickness, w_{Uo}; an inner wall extending between an inner semicircle of the U-shaped portion and the hydrocarbon channel and having a thickness, w_{Ui}, wherein a ratio of w_{Uo} to w_{Ui} is within greater than or equal to 1.2 and less than or equal to

$$2 \times {( \frac{D + d}{D - d} )}^{2} ,$$

where D is a distance between centerlines of the plurality of straight portions and d is a tube diameter, wherein the current voltage source is electrically connected to the electrically conductive tube.

Specific embodiments will now be described with references to the figures.

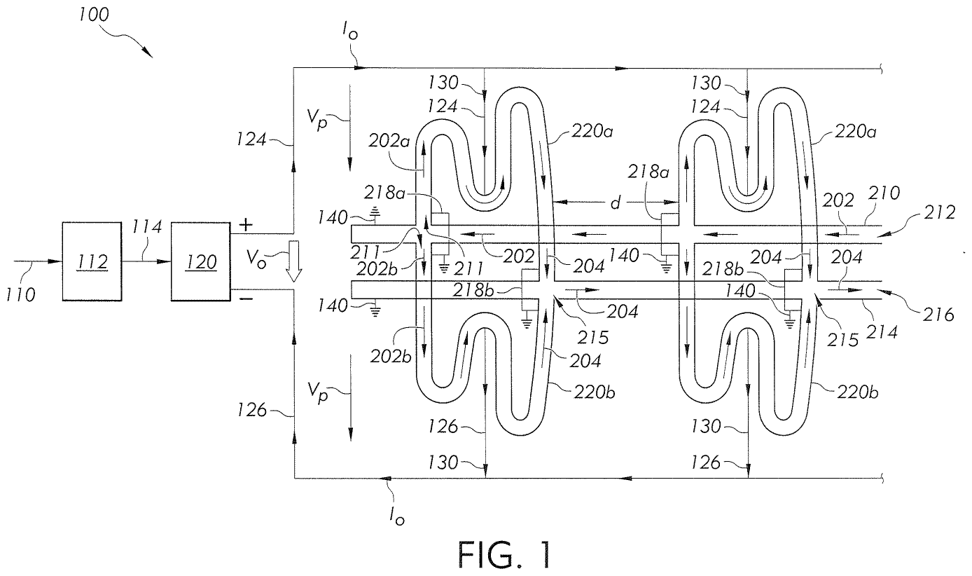

FIG. 1 schematically depicts a heating apparatus 100 for upgrading hydrocarbons from a hydrocarbon fluid 202, according to embodiments described herein.

The heating apparatus 100 includes a pair of electrically conductive tubes (as shown in FIG. 1, the pair may include 220a and 220b), a feed channel 210, a product channel 214, and a current voltage source 120. It is contemplated that the current voltage source 120 may be DC or AC. In the embodiment of heating apparatus 100, the current voltage source 120 is DC. The feed channel 210 has a fluid entrance 212 and a grounded connection 140 and is fluidly connected to inlets 211 of the pair of electrically conductive tubes 220a, 220b. Similarly, the product channel 214 has a fluid exit 216 and a grounded connection 140 and is fluidly connected to outlets 215 of the pair of electrically conductive tubes 220a, 220b.

In embodiments, a power feed 110 may be provided to a transformer 112. The transformer 112 may be any transformer known in the art that transfers electrical energy from one electrical circuit to another circuit. In embodiments, the transformer 112 may be a power step-down transformer that decreases the voltage level of the power to form a decreased voltage power 114 that is provided to the DC current voltage source 120. A power step-down transformer 112 and an associated DC current voltage source 120 may use semiconductor technology such as Thyristor, Diode, Insulated-gate Bipolar Transistor (IGBT), Integrated Gate-Commutated Thyristor (IGCT), which are commercially available by companies such as Fuji Electric or ABB and are commonly used by the electro-chemical or metals industries. It should be understood that although the transformer 112 and the DC current voltage source 120 are depicted for clarity as separate units in FIG. 1, in embodiments these components may be integrated into a single unit.

As shown in FIG. 1, the pair of electrically conductive tubes includes a first electrically conductive tube 220a and a second electrically conductive tube 220b connected in series. As used throughout this disclosure, 220a and 220b will be used to refer to the terms “first electrically conductive tube” and “second electrically conductive tube,” respectively, as to refer to a pair of electrically conductive tubes or to each component of a pair of electrically conductive tubes; however, this is not meant to limit to specifically one pair of electrically conductive tubes or to a first pair of electrically conductive tubes, rather, this is meant to refer to any pair or component of a pair of electrically conductive tubes in general. In embodiments (as shown in FIG. 1), there may be additional pairs of electrically conductive tubes, such as a second pair including 220a and 220b. In embodiments (not shown), there may be only 1 electrically conductive tube or there may be more than 2 pairs of electrically conductive tubes. In embodiments, the heating apparatus 100 may include from 1 to 100 pairs, from 2 to 75 pairs, from 2 to 60 pairs, from 2 to 50 pairs, from 2 to 40 pairs, from 2 to 35 pairs, from 2 to 30 pairs, from 2 to 25 pairs, from 2 to 20 pairs, from 2 to 15 pairs, from 2 to 10 pairs, from 2 to 5 pairs, from 5 to 100 pairs, from 5 to 75 pairs, from 5 to 60 pairs, from 5 to 50 pairs, from 5 to 40 pairs, from 5 to 35 pairs, from 5 to 30 pairs, from 5 to 25 pairs, from 5 to 20 pairs, from 5 to 15 pairs, from 5 to 10 pairs, from 10 to 100 pairs, from 10 to 75 pairs, from 10 to 60 pairs, from 10 to 50 pairs, from 10 to 40 pairs, from 10 to 35 pairs, from 10 to 30 pairs, from 10 to 25 pairs, from 10 to 20 pairs, from 10 to 15 pairs, from 15 to 100 pairs, from 15 to 75 pairs, from 15 to 60 pairs, from 15 to 50 pairs, from 15 to 40 pairs, from 15 to 35 pairs, from 15 to 30 pairs, from 15 to 25 pairs, from 15 to 20 pairs, from 20 to 100 pairs, from 20 to 75 pairs, from 20 to 60 pairs, from 20 to 50 pairs, from 20 to 40 pairs, from 20 to 35 pairs, from 20 to 30 pairs, from 20 to 25 pairs, from 25 to 100 pairs, from 25 to 75 pairs, from 25 to 60 pairs, from 25 to 50 pairs, from 25 to 40 pairs, from 25 to 35 pairs, from 25 to 30 pairs, from 30 to 100 pairs, from 30 to 75 pairs, from 30 to 60 pairs, from 30 to 50 pairs, from 30 to 40 pairs, from 30 to 35 pairs, from 35 to 100 pairs, from 35 to 75 pairs, from 35 to 60 pairs, from 35 to 50 pairs, from 35 to 40 pairs, from 40 to 100 pairs, from 40 to 75 pairs, from 40 to 60 pairs, from 40 to 50 pairs, from 50 to 100 pairs, from 50 to 75 pairs, from 50 to 60 pairs, from 60 to 100 pairs, from 60 to 75 pairs, or from 75 to 100 pairs of electrically conductive tubes. In embodiments where the heating apparatus 100 includes at least 2 pairs of electrically conductive tubes, a first pair of electrically conductive tubes and a second pair of electrically conductive tubes may be physically arranged in parallel to each other separated by distance (d) on a horizontal axis, as shown.

In embodiments, the first electrically conductive tube, the second electrically conductive tube, or both may have a curve along the length of the electrically conductive tube. The shape of each electrically conductive tube can vary to resemble, for example, the letter M, W, U, inverted U, I, or combinations thereof. The number of curves and resultant shapes is dependent on the number of U-bends dictated by the required overall reactor tube length. It is desirable for each electrically conductive tube connected to the power feed 110 to have similar shape and geometries and made with similar materials and having electrical resistance value within plus or minus 10% of a specified range for new materials at room temperature and at operating temperature, to ensure the power output required for each electrically conductive tube is within a desirable range of approximately +/−11%, thus ensuring the heat flux to all conduits are within a narrower range (plus or minus 11% of mean value) compared to heat fluxes delivered to conduits in existing conventional combustion fired furnaces (which can vary by as much as 100% across an entire furnace). The uniform heat flux of the electrically conductive tubes provides a more uniform conduit temperature such that the maximum conduit temperature—both along a single conduit and across neighboring conduits in the furnace—is reduced compared to the maximum conduit temperature found in conventional combustion furnaces. A reduction in the maximum conduit temperature can permit an increase in the average conduit temperature, which will improve the conversion of reactants and increase the selectivity to desired products (ethylene). It follows that the improved control of the maximum temperature of conductive cracking conduits will result in lower rates of coke build-up on the inner conduit wall, and lower rates of carbon diffusion into the coil material, commonly known as carburization. Conduits that operate free of coke buildup exhibit higher heat transfer rates contribute to conduit cooling, further limiting conduit overheating. Excessive material carburization can affect coil mechanical integrity and lifetime. Reduced coking rates leads to longer operational runtimes, decreasing the frequency of costly furnace shutdowns needed for de-coking of the conduits, and hence longer overall conduit lifetimes can be expected from use of electrically conductive tubes.

In embodiments, any pair of electrically conductive tubes may have an electric resistivity of from 1.0 to 4.0μΩ·m at 900° C., from 1.0 to 3.5μΩ·m at 900° C., from 1.0 to 3.0μΩ·m at 900° C., from 1.0 to 2.5μΩ·m at 900° C., from 1.0 to 2.0μΩ·m at 900° C., from 1.0 to 1.5μΩ·m at 900° C., from 1.5 to 4.0μΩ·m at 900° C., from 1.5 to 3.5μΩ·m at 900° C., from 1.5 to 3.0μΩ·m at 900° C., from 1.5 to 2.5μΩ·m at 900° C., from 1.5 to 2.0μΩ·m at 900° C., from 2.0 to 4.0μΩ·m at 900° C., from 2.0 to 3.5μΩ·m at 900° C., from 2.0 to 3.0μΩ·m at 900° C., from 2.0 to 2.5μΩ·m at 900° C., from 2.5 to 4.0μΩ·m at 900° C., from 2.5 to 3.5μΩ·m at 900° C., from 2.5 to 3.0μΩ·m at 900° C., from 3.0 to 4.0μΩ·m at 900° C., from 3.0 to 3.5μΩ·m at 900° C., or from 3.5 to 4.0μΩ·m at 900° C. In embodiments, the electric resistivity of an electrically conductive tube may vary over the length of an electrically conductive tube.

In embodiments, any electrically conductive tube may have an inner diameter of from 1 to 6 inches (in), from 1 to 5 in, from 1 to 4 in, from 1 to 3 in, from 1 to 2 in, from 2 to 6 in, from 2 to 5 in, from 2 to 4 in, from 2 to 3 in, from 3 to 6 in, from 3 to 5 in, from 3 to 4 in, from 4 to 6 in, from 4 to 5 in, or from 5 to 6 in. Any electrically conductive tube may have a wall thickness of from 0.1 to 1.5 in, from 0.1 to 1.25 in, from 0.1 to 1.0 in, from 0.1 to 0.75 in, from 0.1 to 0.5 in, from 0.1 to 0.25 in, from 0.25 to 1.5 in, from 0.25 to 1.25 in, from 0.25 to 1.0 in, from 0.25 to 0.75 in, from 0.25 to 0.5 in, from 0.5 to 1.5 in, from 0.5 to 1.25 in, from 0.5 to 1.0 in, from 0.5 to 0.75 in, from 0.75 to 1.5 in, from 0.75 to 1.25 in, from 0.75 to 1.0 in, from 1.0 to 1.5 in, from 1.0 to 1.25 in, or from 1.25 to 1.5 in. Any electrically conductive tube may have a length of 10 to 60 meters (m), from 10 to 50 m, from 10 to 40 m, from 10 to 30 m, from 10 to 20 m, from 20 to 60 m, from 20 to 50 m, from 20 to 40 m, from 20 to 30 m, from 30 to 60 m, from 30 to 50 m, from 30 to 40 m, from 40 to 60 m, from 40 to 50 m, or from 50 to 60 m. In embodiments, the inner diameter, the wall thickness, or both may vary over the length of an electrically conductive tube.

As referenced previously, various properties of the electrically conductive tubes may be modified to deliver different heat loads to different sections of the electrically conductive tube. It is contemplated that modifying the properties of the electrically conductive tube may result in delivering heat in a way that facilitates improved performance. Tailoring the power and heat load delivered to an electrically conductive tube may be accomplished by adjusting the electrical resistance of a first portion of an electrically conductive tube and the electrical resistance of a second portion of an electrically conductive tube relative to each other. In embodiments, the first portion of any electrically conductive tube may be proximate to the inlet 211. Similarly, the second portion of any electrically conductive tube may be proximate to the outlet 215. The electrical resistance can be adjusted by adjusting the properties of the portions of the electrically conductive tube, where the properties are any of those described herein. It is contemplated that the resistance of each portion of an electrically conductive tube may also be changed by using different materials having different electrical resistivity. Additionally, it is also contemplated that resistance may be changed by using different cross-sectional area in different sections of the tube length.

In embodiments, the first electrically conductive tube 220a may be electrically connected to the second electrically conductive tube 220b. In embodiments, a first current bridge link 218a and a second current bridge link 218b are electrically positioned between the first electrically conductive tube 220a and the second electrically conductive tube 220b. The current bridge links 218a, 218b are configured to conduct current across the feed channel 210, the product channel 214, or both. In embodiments, the current bridge links 218a, 218b may be connected to a grounded connection 140, as shown in FIG. 1 where a grounded connection 140 is shown connected to the current bridge link 218a. Although FIG. 1 depicts the grounded connection 140 connected to the current bridge link 218a, it should be understood that there may be additional or alternative grounded connections 140 connected to the other current bridge links 218a, 218b. In embodiments (not shown), each current bridge link 218a, 218b may be connected to a separate or independent grounded connection 140.

The first and second current bridge links 218a, 218b may include materials possessing high melting point, low electrical resistance and are galvanically compatible with the material of the conduits to which they are attached, such as the same material as the conduit. According to embodiments, the first and second current bridge links 218a, 218b are made from material that has an electrical resistivity that is less than or equal to the resistivity of the material used to form the pair of electrically conductive tubes 220a, 220b, to limit excessive heat generated in the current bridge links.

The DC current voltage source 120 is electrically connected to the first electrically conductive tube 220a and the second electrically conductive tube 220b. In embodiments, the pair of electrically conductive tubes 220a, 220b is configured such that DC current 130 generated from the DC current voltage source 120 flows from the first electrically conductive tube 220a to the second electrically conductive tube 220b and from the second electrically conductive tube 220b to the DC current voltage source 120. In embodiments, the DC current 130 may flow from the first electrically conductive tube 220a to the second electrically conductive tube 220b through the first and second current bridge links 218a, 218b.

The DC current voltage source 120 has an output voltage (Vₒ) twice that of the voltage potential (Vₚ) of each electrically conductive tube thus allowing the same amount of DC current 130 to power the pair of electrically conductive tubes 220a, 220b, electrically connected in series. It is contemplated that having the output voltage (Vₒ) be twice that of the voltage potential (Vₚ) of each electrically conductive tube allows the output current (Iₒ) from the DC current voltage source 120 for the pair of electrically conductive tubes 220a, 220b equal to the current of each pipe assembly. This maintains the output current (Iₒ) from the DC current voltage source 120 at a minimum level while allowing the inlets 211 and outlets 215 of the pair of electrically conductive tubes 220a, 220b to be electrically grounded to earth ground through the grounded connection 140 to ensure safe operation while also not requiring the use of electrically isolating means (such as isolation flanges).

As previously stated, the pair of electrically conductive tubes 220a, 220b have inlets 211 and outlets 215. Any electrically conductive tube may have one inlet 211 (as shown) or may have multiple inlets 211 (not shown). In embodiments, any electrically conductive tube may have 2, 3, 4, 5, or 6 inlets 211. Any electrically conductive tube may have one outlet 215 (as shown) or may have multiple outlets 215 (not shown). In embodiments, any electrically conductive tube may have 2, 3, 4, 5, or 6 outlets 215. In embodiments, the inlets 211 of the pair of electrically conductive tubes 220a, 220b each have 0 voltage potential. Additionally or alternatively, in embodiments, the outlets 215 of pair of electrically conductive tubes 220a, 220b each have 0 voltage potential. The inlets 211 and/or the outlets 215 may have 0 voltage potential due to being electrically conductively connected to the grounded connection 140.

The heating apparatus 100 may be further configured to send DC current 130 from the inlet 211 of the first electrically conductive tube 220a across the first current bridge link 218a to the inlet 211 of the second electrically conductive tube 220b. The heating apparatus 100 may be further configured to send DC current 130 from the outlet 215 of the first electrically conductive tube 220a across the second current bridge link 218b to the outlet 215 of the second electrically conductive tube 220b. It is contemplated that the current bridge links 218a, 218b are low resistance paths as compared to the connections between the inlets 211 or the outlets 215; therefore a relatively larger portion of the DC current 130 flows through the current bridge links 218a, 218b than through the connections between the inlets 211 and the outlets 215.

In embodiments, the heating apparatus 100 may further include a positive conductor 124 and a negative conductor 126. In embodiments, the positive conductor 124 electrically connects the DC current voltage source 120 and the first electrically conductive tube 220a. Similarly, the negative conductor 126 may electrically connect the DC current voltage source 120 and the second electrically conductive tube 220b.

In embodiments, the heating apparatus 100 may be configured to send DC current 130 from the DC current voltage source 120 through the positive conductor 124 to the first electrically conductive tube 220a. Additionally or alternatively, the heating apparatus 100 may be configured to send DC current 130 from the second electrically conductive tube 220b through the negative conductor 126 to the DC current voltage source 120.

In embodiments, the positive conductor 124 may be electrically connected to a position of the first electrically conductive tube 220a between the inlet 211 and outlet 215. Similarly, in embodiments, the negative conductor 126 may be electrically connected to a position of the second electrically conductive tube 220b between the inlet 211 and outlet 215. In embodiments, the positive conductor 124 and the negative conductor 126 may be electrically connected at positions approximately halfway along the length of the electrically conductive tube 220a, 220b. In embodiments, the positive conductor 124 and the negative conductor 126 may be electrically connected at positions relatively closer to the inlet 211 than the outlet 215 of the electrically conductive tube 220a, 220b, as shown in FIG. 1. In embodiments where the positive conductor 124 and the negative conductor 126 are electrically connected at positions relatively closer to the inlet 211 than the outlet 215 along the length of the electrically conductive tube 220a, 220b, greater than 50% of the total electrically generated heat per electrically conductive tube 220a, 220b, may be generated in a first half of the electrically conductive tube 220a, 220b. In embodiments, from 50% to 95%, from 50% to 90%, from 50% to 85%, from 50% to 80%, from 50% to 75%, from 50% to 70%, from 50% to 65%, from 50% to 60%, from 50% to 55%, from 55% to 95%, from 55% to 90%, from 55% to 85%, from 55% to 80%, from 55% to 75%, from 55% to 70%, from 55% to 65%, from 55% to 60%, from 60% to 95%, from 60% to 90%, from 60% to 85%, from 60% to 80%, from 60% to 75%, from 60% to 70%, from 60% to 65%, from 65% to 95%, from 65% to 90%, from 65% to 85%, from 65% to 80%, from 65% to 75%, from 65% to 70%, from 70% to 95%, from 70% to 90%, from 70% to 85%, from 70% to 80%, from 70% to 75%, from 75% to 95%, from 75% to 90%, from 75% to 85%, from 75% to 80%, from 80% to 95%, from 80% to 90%, from 80% to 85%, from 85% to 95%, from 85% to 90%, or from 90% to 95% of the total electrically generated heat per electrically conductive tube 220a, 220b, may be generated in a first half of the electrically conductive tube 220a, 220b. In embodiments, the average heat load (total heat divided by the total surface area) along the length of each electrically conductive tube ranges from 10 to 150 kilowatt per square meter (kW/m²), such as from 15 to 150 Kw/m², from 20 to 150 Kw/m², from 25 to 150 Kw/m², from 30 to 150 Kw/m², from 40 to 150 Kw/m², from 50 to 150 Kw/m², from 70 to 150 Kw/m², from 90 to 150 Kw/m², from 100 to 150 Kw/m², from 125 to 150 Kw/m², from 10 to 125 kW/m², from 15 to 125 Kw/m², from 20 to 125 Kw/m², from 25 to 125 Kw/m², from 30 to 125 Kw/m², from 40 to 125 Kw/m², from 50 to 125 Kw/m², from 70 to 125 Kw/m², from 90 to 125 Kw/m², from 100 to 125 Kw/m², from 10 to 100 kW/m², from 15 to 100 Kw/m², from 20 to 100 Kw/m², from 25 to 100 Kw/m², from 30 to 100 Kw/m², from 40 to 100 Kw/m², from 50 to 100 Kw/m², from 70 to 100 Kw/m², from 90 to 100 Kw/m², from 10 to 90 kW/m², from 15 to 90 Kw/m², from 20 to 90 Kw/m², from 25 to 90 Kw/m², from 30 to 90 Kw/m², from 40 to 90 Kw/m², from 50 to 90 Kw/m², from 70 to 90 Kw/m², from 10 to 70 kW/m², from 15 to 70 Kw/m², from 20 to 70 Kw/m², from 25 to 70 Kw/m², from 30 to 70 Kw/m², from 40 to 70 Kw/m², from 50 to 70 Kw/m², from 10 to 50 kW/m², from 15 to 50 Kw/m², from 20 to 50 Kw/m², from 25 to 50 Kw/m², from 30 to 50 Kw/m², from 40 to 50 Kw/m², from 10 to 40 kW/m², from 15 to 40 Kw/m², from 20 to 40 Kw/m², from 25 to 40 Kw/m², from 30 to 40 Kw/m², from 10 to 30 kW/m², from 15 to 30 Kw/m², from 20 to 30 Kw/m², from 25 to 30 Kw/m², from 10 to 25 kW/m², from 15 to 25 Kw/m², from 20 to 25 Kw/m², from 10 to 20 kW/m², from 15 to 20 Kw/m², or from 10 to 15 kW/m².

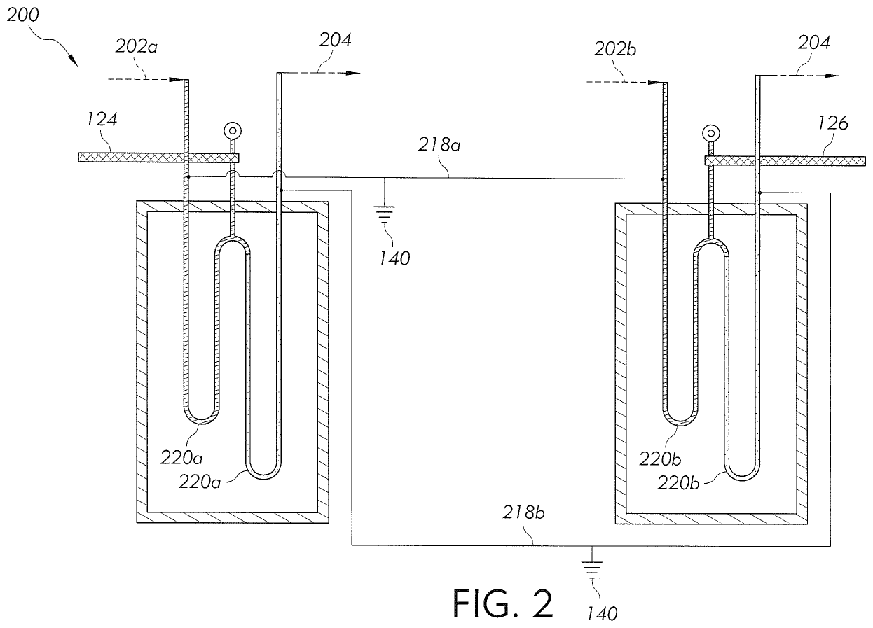

The above heating apparatus 100 according to embodiments disclosed and described herein will now further be defined with reference to FIG. 2, which is a side view of the heating apparatus 100 according to one or more embodiments. As shown in FIG. 2, the heating apparatus 200 comprises grounded connections 140 that are electrically connected to the left and right side the first electrically conductive tube 220a and second electrically conductive tube 220b. A positive conductor 124 is connected to the middle of the first electrically conductive tube 220a to provide an electrical current to the first electrically conductive tube 220a. A negative conductor 126 is connected to the middle of the second electrically conductive tube 220b to provide an electrical circuit with the first electrically conductive tube 220a and the positive conductor 124. In embodiments, an electrical current may flow from the first electrically conductive tube 220a to the second electrically conductive tube 220b through the first and second current bridge links 218a, 218b.

As a result of the electrical resistance of the first electrically conductive tube 220a, applying electrical current via the positive conductor 124 to the first electrically conductive tube 220a causes the temperature of the first electrically conductive tube 220a to increase, thereby heating the first portion of the hydrocarbon fluid 202a that enters the first electrically conductive tube 220a of the heating apparatus 200. The first portion of the hydrocarbon fluid 202a reacts within the first electrically conductive tube 220a to form a product stream 204. Additionally, as a result of the electrical resistance of the second electrically conductive tube 220b, applying electrical current via the circuit formed by the positive conductor 124, the first electrically conductive tube 220a, the second electrically conductive tube 220b, and the negative conductor 126 causes the temperature of the second electrically conductive tube 220b to increase, thereby heating the second portion of the hydrocarbon fluid 202b that enters the second electrically conductive tube 220b of the heating apparatus 200. The second portion of the hydrocarbon fluid 202b reacts within the second electrically conductive tube 220b to form a product stream 204.

### Baseline Design

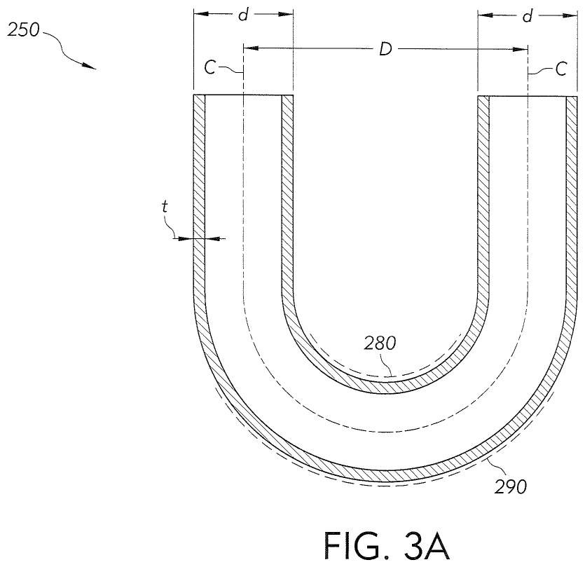

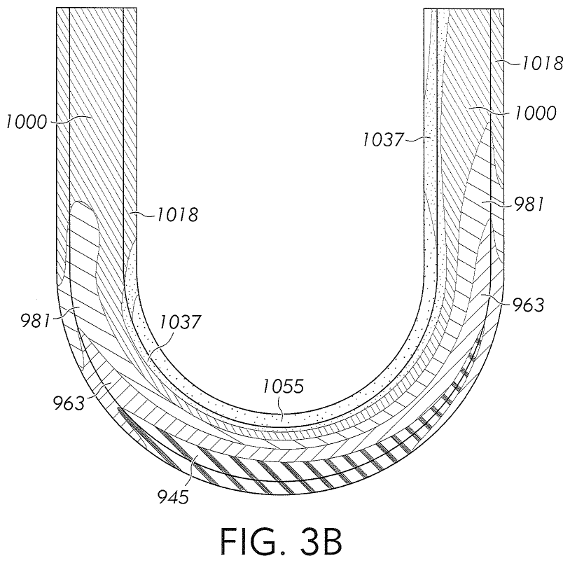

Referring now to FIG. 3A, a single U-bend 250 is depicted comprising an inner semi-circle 280 and an outer semi-circle 290 of the U-bend metal wall. The tube thickness t is uniform throughout the tube, including the U-bend. This design will hereinafter be referred to as the “Baseline Design.” The U-bend 250 comprises D, a distance between the centerlines C of the plurality of straight portions of a hydrocarbon channel. The U-bend 250 also comprises tube diameter d. The inner semicircle 280 and outer semicircle 290 of the U-bend 250 form electric circuits, each with different length, and subsequently, with different electric resistance.

FIG. 3B depicts the temperature profile of the Baseline Design of FIG. 3A. The resulting electric current density and joule heating power differ from the inner semicircle to the outer semicircle, which leads to a nonuniform heat flux in the U-bend. Specifically, a U-bend with the same wall thickness at both the inner semicircle 280 and the outer semicircle 290 as the straight legs of the coil leads to a higher joule heating intensity at the inner semicircle 280 of the U-bend. As seen in FIG. 3B, the temperature at the inner semicircle 280 for the Baseline Design is over 1050° C., while the outer semicircle 290 is slightly less than 950° C. These temperature differences can lead to the formations of hot spots, result in coking, and decrease product yield.

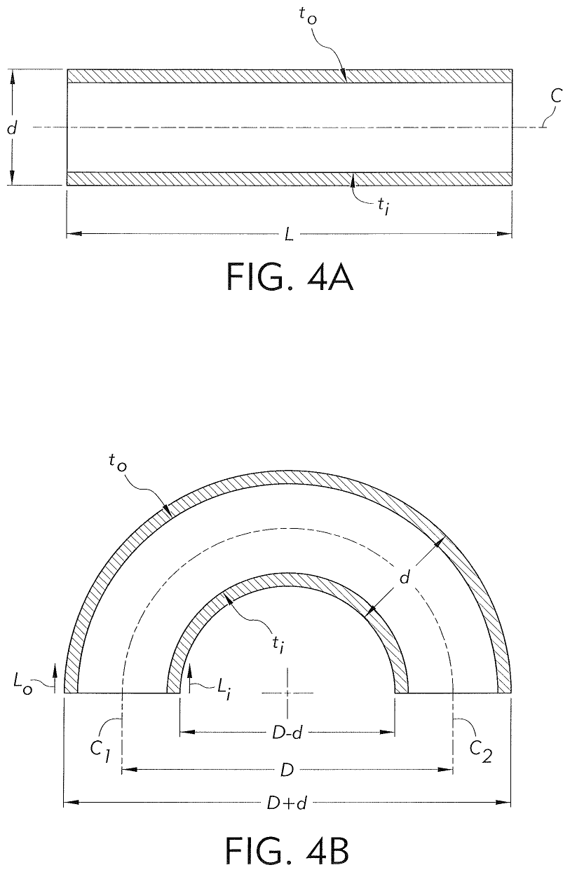

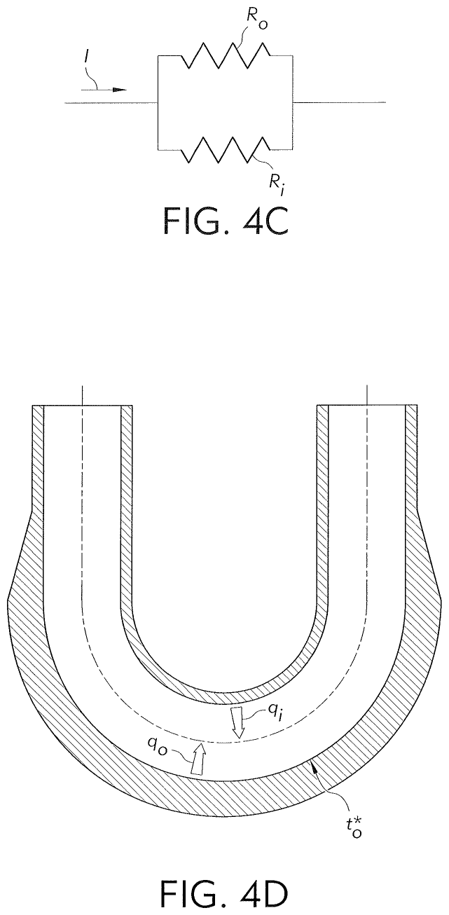

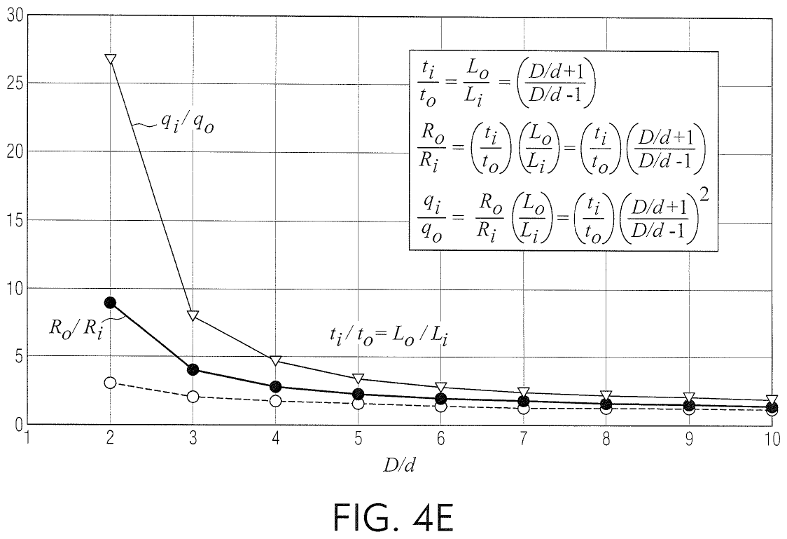

The formation and dimensions of a U-bend will now be described with reference to FIG. 4A and FIG. 4B. A straight pipe from which a U-bend will be formed is depicted in FIG. 4A, and FIG. 4B depicts a U-bend formed from the straight pipe depicted in FIG. 4A.

With reference now to FIG. 4A, the straight pipe has an outer wall tₒ and an inner wall tᵢ that each have the same thickness t. It should be understood that the outer wall tₒ and inner wall tᵢ are for naming and explanation purposes as the pipe is circumferentially enclosed by a single wall. The straight pipe has a diameter d that includes the wall thickness t, and each of the inner wall tᵢ and the outer wall tₒ has the same length L. Finally, a centerline C extends axially through the center of the straight pipe. Because the inner wall tᵢ and the outer wall tₒ have the same thickness (t) and the same length (L) it follows that product of the thickness and length of the inner wall tᵢ and the product of the thickness and length of the outer wall tₒ are equal.

FIG. 4B depicts a U-bend made from the straight pipe depicted in FIG. 4A. With reference now to FIG. 4B, the U-bend includes the outer wall with an outer wall thickness t and an outer wall length Lₒ. The U-bend also includes an inner wall with an inner wall thickness t and an inner wall length Lᵢ. The U-bend also includes a centerline diameter D that extends from one centerline of the U-Bend C₁ to another centerline of the U-bend C₂. Accordingly, the U-bend has an outer diameter that is the sum of the centerline diameter D of the U-bend and the diameter of the pipe d that forms the U-bend (i.e., the outer diameter of the U-bend is equal to D+d). The inner diameter of the U-bend is the difference between the centerline diameter D of the U-bend and that diameter d of the pip that forms the U-bend (i.e., the inner diameter of the U-bend is equal to D−d).

According to embodiments, when the straight pipe as shown in FIG. 4A is bent into a U-Bend as shown in FIG. 4B, the outer wall tₒ will be stretched, such that the thickness of the outer wall tₒ decreases so that it is less than t (depicted in FIG. 4A) and the length of the outer wall Lₒ increases so that it is greater than L (depicted in FIG. 4A). In this way, the thickness of the outer wall tₒ does not equal the thickness of the inner wall tᵢ and the length of the outer wall Lₒ does not equal the length of the inner wall Lᵢ.

Because, as shown in FIG. 4A, the U-bend is formed from a pipe having a uniform length, it follows that the length of the outer wall Lₒ is equal to the outer diameter of the U-bend (i.e., Lₒ=D+d) and the length of the inner wall Lᵢ is equal to the inner diameter of the U-bend (i.e., Lᵢ=D−d). Moreover, because the U-bend is formed from a pipe having a uniform length and uniform wall thickness, it follows that the product of the outer wall thickness tₒ and outer wall length Lₒ is still equal to the product of the inner wall thickness tᵢ and the inner wall length Lᵢ (i.e., tₒ×Lₒ=tᵢ×Lᵢ), as was the case with the straight pipe. Accordingly, the ratio of the wall thicknesses

$$( i . e . , \frac{{t}_{i}}{{t}_{o}} )$$

is equivalent to the ratio of the wall lengths

$$\frac{{L}_{o}}{{L}_{i}} .$$

Thus, both the ratio of the wall thicknesses and ratio of the wall lengths are therefore equal to

$$( \frac{D + d}{D - d} ) .$$

Written differently,

$$\frac{{t}_{i}}{{t}_{o}} = \frac{Lo}{Li} = ( \frac{D + d}{D - d} ) .$$

Referring now to FIG. 4C, the electrical resistances of the U-bend are shown. The total current flows over two circuits. The resistance along the outer bend is denoted by Rₒ and the resistance along the inner bend is denoted by Rᵢ. Because the outer wall Wₒ and the inner wall Wᵢ are made from the same material, the resistance coefficient of the outer wall Wₒ and the inner wall Wᵢ are the same. It therefore follows that the ratio of the resistances are indicated by the following equations

$$\frac{Ro}{Ri} = ( \frac{{t}_{i}}{{t}_{o}} ) ( \frac{Lo}{Li} ) = {( \frac{D + d}{D - d} )}^{2} .$$

Referring now to FIG. 4D, the wall heat fluxes are represented by the inner wall heat flux, (qᵢ) and the outer wall heat flux (qₒ). It follows that the ratio of the heat fluxes

$$( \frac{qi}{qo} ) = ( \frac{Ro}{Ri} ) ( \frac{Lo}{Li} ) = ( \frac{{t}_{i}}{{t}_{o}} ) {( \frac{D + d}{D - d} )}^{2} .$$

Ideally, the heat fluxes would be equal, or

$$( \frac{qi}{qo} ) = 1 = ( \frac{{t}_{i}}{{t}_{o}} ) {( \frac{D + d}{D - d} )}^{2} .$$

To ensure that the heat fluxes are equal, the U-bend inner wall thickness, tᵢ, can be equivalent to the straight wall thickness, t (as noted in FIG. 4B) and the U-bend outer wall thickness, tₒ, will be denoted as t*ₒ. It therefore follows that

$${t}_{o}^{*} = {t}_{i} {( \frac{D + d}{D - d} )}^{2} .$$

Thus, the maldistribution is proportional to

$${( \frac{D + d}{D - d} )}^{2} ,$$

where d is the outside diameter of the electrically conductive tube and D is the diameter of the U-bend centerline. A higher maldistribution in the volumetric Joule heating translates into maldistributions in TMT and hot spots in the U-bend, causing more rapid coke formation and higher risk to tube mechanical integrity.

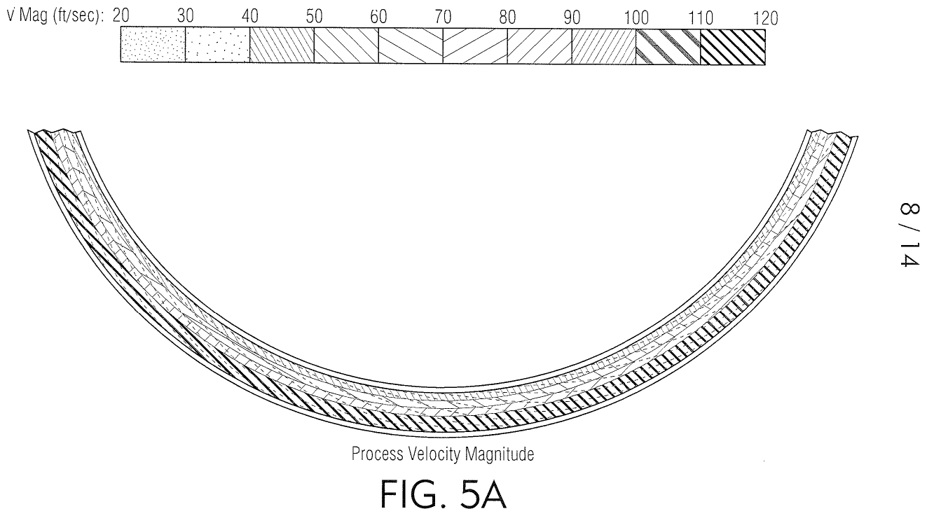

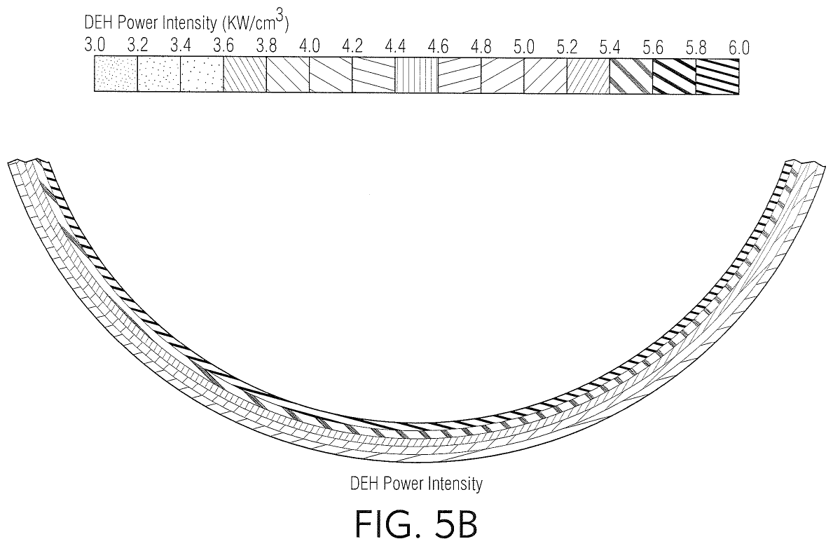

One way to reduce hot spots that occur in the Baseline Design is to use U-bends with a larger U-bend diameter (centerline-to-centerline). To achieve the desired temperature distribution, a tube with a U-bend having a D/d ratio greater than or equal to 20. (i.e. D/d≥20) may be used. A drawback of this approach is that it requires a large footprint of the furnace. FIG. 4E provides a graph that plots the resistance ratios, thickness ratios, and heat flux ratios as a function of the bend diameter ratios. The graph shows that as the bend diameter ratio increases, the resistance ratios, wall thickness ratios and heat flux ratios move closer to 1. For example, for a tube with an outer diameter of four inches, the corresponding U-bend diameter would have to be greater than or equal to 80 inches, and a W-shaped tube would be greater than or equal to 240 inches. As another example, a tube with an outer diameter of 6 inches would have a corresponding U-bend diameter greater than or equal to 120 inches, and a W-shaped tube would be greater than or equal to 360 inches. These diameters are larger than the conventional design of most cracking furnaces and would require additional retrofitting and cost to incorporate. As a non-limiting example, bend diameters of coils in existing conventional furnaces are in the range of 3<D/d<5. However, it is contemplated that bend diameters in furnaces could be at least greater than or equal to 2 and less than or equal to 20. For example, the bend diameters may have a ratio greater than or equal to 2, greater than or equal to 4, greater than or equal to 6, greater than or equal to 8, or greater than or equal to 10. The bend diameters may also have a ratio less than or equal to 20, less than or equal to 18, less than or equal to 16, less than or equal to 14, less than or equal to 12, or less than or equal to 10. The bend diameters may also have a ratio of from 2 to 20, from 2 to 19, from 2 to 18, from 2 to 17, from 2 to 16, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4, from 2 to 3, from 3 to 20, from 3 to 19, from 3 to 18, from 3 to 17, from 3 to 16, from 3 to 15, from 3 to 14, from 3 to 13, from 3 to 12, from 3 to 11, from 3 to 10, from 3 to 9, from 3 to 8, from 3 to 7, from 3 to 6, from 3 to 5, from 3 to 4, from 4 to 20, from 4 to 19, from 4 to 18, from 4 to 17, from 4 to 16, from 4 to 15, from 4 to 14, from 4 to 13, from 4 to 12, from 4 to 11, from 4 to 10, from 4 to 9, from 4 to 8, from 4 to 7, from 4 to 6, or even from 4 to 5. Another drawback of the larger U-bend diameter proposed above is that a larger U-bend diameter is unfavorable to heat transfer and TMT. Referring now to FIG. 5A, the process gas passing though the U-bend tends to have higher velocity toward the outer semicircle of the U-bend and lower velocity toward the inner semicircle of the U-bend. On the other hand, as depicted in FIG. 5B, the electrically generated heat flux is higher at the inner semicircle and lower at the outer semicircle of the U-bend. As seen in FIGS. 5A and 5B, the higher-process-velocity region and the higher-joule-heating region diverge. This divergence leads to unfavorable hot spots and faster coke formation rate for the inner portion of the U-bend and lower heat transfer efficiency for the outer portion of the U-bend.

In sum, to avoid large process footprints, it is desirable that the U-bend accounts for a small fraction of the total tube volume. Accordingly, U-bends with a large diameter do not fulfill the desire for a small volumetric fraction. Furthermore, regardless of the volumetric preferences of the U-bends, the provision of a large U-bend semicircle does not fully mitigate heating non-uniformities, as seen in FIGS. 5A, and 5B. Accordingly, a new design for U-bends in electrically heated cracking furnace that eliminates hot spots and improves heat transfer efficiency is needed.

### Design A

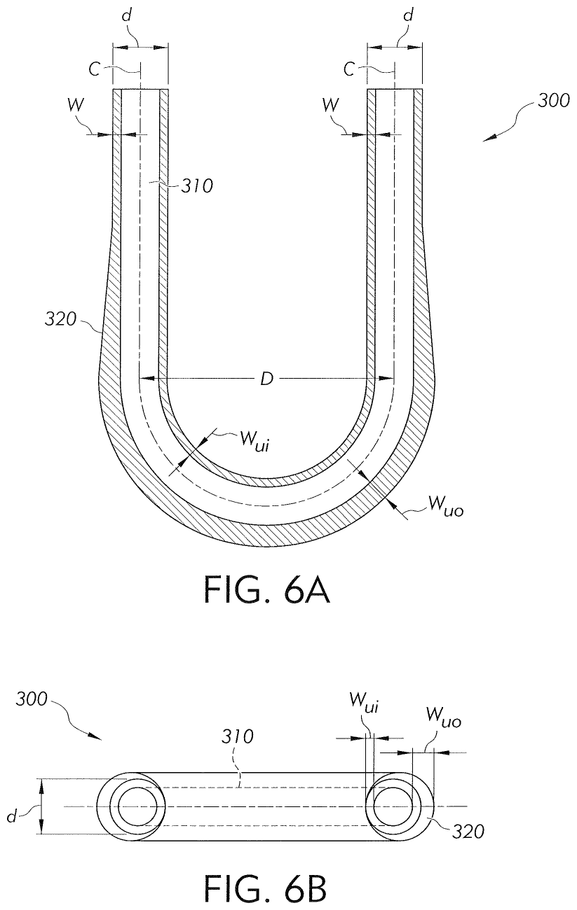

FIG. 6A discloses a front view of a U-bend 300 design that provides a continuous heat profile and reduces heat flux and temperature non-uniformities across the U-bend. This design will hereinafter be referred to as “Design A.” Design A comprises an inner process tube 310 of uniform diameter and an outer tube 320. FIG. 5A depicts a Design A U-bend 300 where the electrical resistance along the U-shaped portion of the electrically conductive tube is circumferentially more non-uniform than the electrical resistance along the straight portions of the electrically conductive tube. Design A facilitates a more circumferentially uniform electric current by gradually increasing the thickness of U-bend as moving from the inner semi-circle to the outer semi-circle, and when the inner and outer semi-circles form an electric circuit, the total electrical resistance is better balanced along the inner and outer semi-circles. Accordingly, Design A is unlike the U-bend the Baseline Design. In the Baseline Design, the electric resistance of the inner semicircle of the U-bend is higher than that of the outer semicircle such that the resultant joule heating heat flux around the U-bend circumference is balanced.

Referring back to Design A, in FIG. 6A, the U-shaped portion of the electrically conductive tube 300 comprises an inner semicircle having a first tube wall thickness (w_{Ui}) and an outer semicircle having a second tube wall thickness (w_{Uo}). A ratio of w_{Uo} to w_{Ui} is greater than or equal to 1.2 and less than or equal to

$$2 \times {( \frac{D + d}{D - d} )}^{2} ,$$

where D is a distance between the centerlines C of the plurality of straight portions of a hydrocarbon channel and d is a tube diameter of the electrically conductive tube 320. Preferably, the ratio of w_{Uo} to

$${w}_{Ui} is within \pm 30 \% of {( \frac{D + d}{D - d} )}^{2} ,$$

or, more preferably, the ratio of w_{Uo} to

$${w}_{Ui} is within \pm 10 \% of {( \frac{D + d}{D - d} )}^{2} ,$$

The U-bend 300 has a straight leg tube wall thickness of w. The thickness of the tube may gradually taper from the U-bend to the straight leg. However, it is contemplated that the thickness of the tube may instantaneously increase such that the tube thickness is w at one instant and w_{Ui} or w_{Uo} at the next instant.

FIG. 6B depicts the top view of the U-bend design of FIG. 6A. As seen in FIG. 6B, the inner wall thickness (w_{Ui}) is smaller than the outer wall thickness (w_{Uo}). The increased wall thickness at the outer semicircle provides an increased electrical conductivity of the wall along the outer semicircle. Accordingly, this allows for better balance of heat along the inner semicircle and outer semicircle of the U-bend.

### Design B

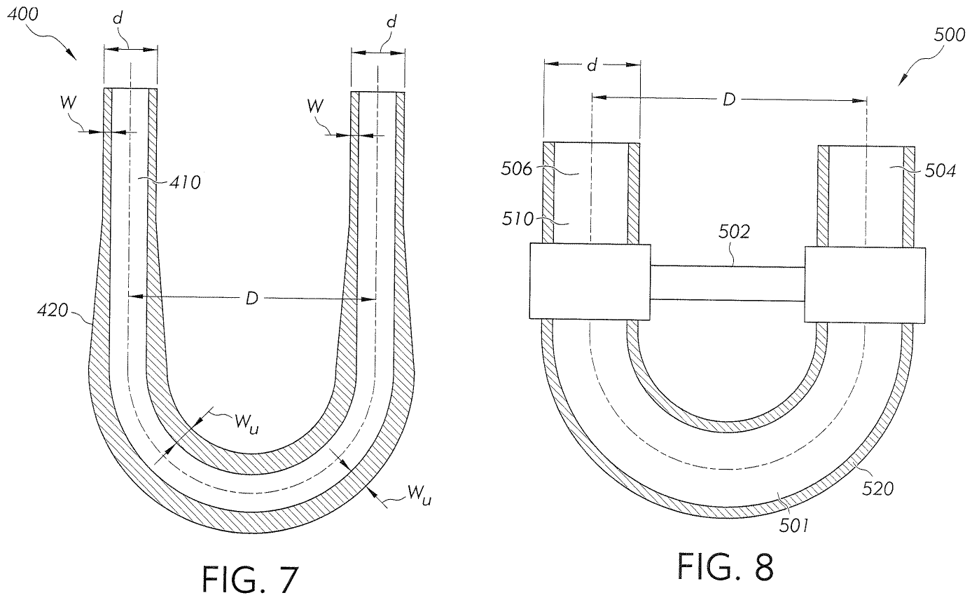

FIG. 7 discloses an alternative to the U-bend geometry disclosed in FIG. 6A and FIG. 6B. The design in FIG. 7 will hereinafter be referred to as “Design B.” Design B comprises an inner process tube 410 of uniform diameter and an outer tube 420. Design B also comprises D, a distance between the centerlines C of the plurality of straight portions of a hydrocarbon channel and d as a tube diameter of the electrically conductive tube 420. In FIG. 7, the Design B U-bend 400 has a uniform tube wall thickness (wᵤ) that is 1.25 to 5 times of the straight leg tube wall thickness (w), such that 1.25w≤wᵤ≤5w. In embodiments, the uniform tube wall thickness (wᵤ) may be from 1.25w to 5w, 1.50w to 5w, 1.75w to 5w, 2.00w to 5w, 2.25w to 5w, 2.50w to 5w, 2.75w to 5w, 3.00w to 5w, 3.25w to 5w, 3.50w to 5w, 3.75w to 5w, 4.00w to 5w, 4.25w to 5w, 4.50w to 5w, or from 4.75w to 5w. Like Design A, the tube thickness of Design B may gradually taper from the U-bend to the straight leg. However, it is contemplated that the thickness of the tube may instantaneously increase such that the tube thickness is w at one instant and w_{U} at the next instant.

### Design C

FIG. 8 discloses another alternative to the geometries disclosed in FIGS. 6A, 6B and 7. The design depicted in FIG. 8 will hereinafter be referred to as “Design C.” Design C comprises a U-bend 500 that comprises an inner process tube 510 of uniform diameter and an outer tube of uniform diameter 520 to form a U-shaped portion of the electrically conductive tube 501 that form two straight legs of the tube, 504 and 506. Design C also comprises D, a distance between the centerlines of the plurality of straight portions of a hydrocarbon channel, and d as a tube diameter of the electrically conductive tube 520. The U-bend 501 is short-circuited using an electric conductive bridge 502 whose electric resistance is less than half of the electric resistance of the U-bend. The bridge 502 may be located along various points of the straight legs 504 and 506. The bridge 502 may be fastened to the straight legs 504 and 506 via a clasp or a weld. Other methods of fastening the bridge 502 to the straight legs 504 and 506 are contemplated and are considered within the scope of this disclosure.

It should be understood that the system shown in FIG. 8 is exemplary only and is not meant to be limiting on this disclosure; numerous other systems may be utilized within the scope of this disclosure. The electrical coupler may be made of any material that the conductor is made of. The material may be, but is not limited to, a nickel-rich alloy, a chrome-rich alloy, or aluminum alloy. The electrical coupler may be selected from a braided strap, a metallic bar, or the like. The electrical coupler may be flexible or rigid. The electrical coupler may also include flexible joints to minimize mechanical stresses induced by the thermal expansion. For example, the electrical coupler may be a flexible, metallic, electrically conducting cable.

In embodiments, any electrical coupler may have an electric resistivity of from 0.2 to 1.5. For example the electrical coupler may have an electric resistivity of from 0.2 μΩ·m at 900° C. to 0.5μΩ·m at 900° C., from 0.5μΩ·m at 900° C. to 1.0, or from 1.0μΩ·m at 900° C. to 1.5μΩ·m at 900° C.

In embodiments, Design C may have an alternative design such that the straight legs 504 and 506 are connected to individual electrically conductive bridges. In embodiments, an electrical current may flow from a first straight leg 504 to the second straight leg 506 through a first current bridge link and a second current bridge link.

### EXAMPLES

A process for upgrading hydrocarbons was modeled using a computational fluid dynamics (CFD) model with an ethane feed steam cracking process tube.

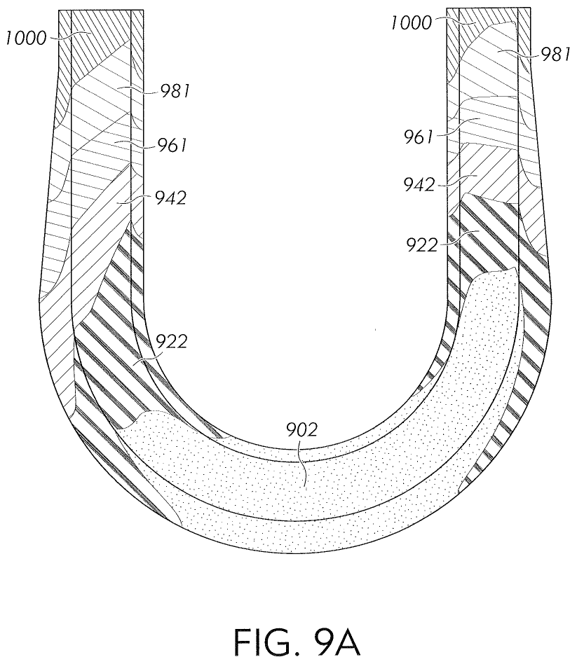

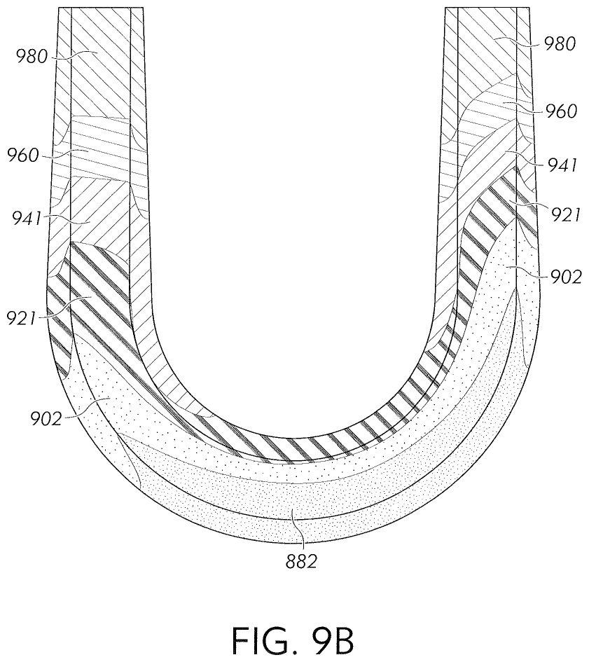

FIG. 9A and FIG. 9B along with FIG. 3B summarize the three CFD modeling cases. As described hereinabove, FIG. 3B depicts the Baseline Design case where the U-bend wall thickness was the same as that of the straight legs. Design A in FIG. 9B depicts the case where the tube wall thickness of the U-bend gradually increased from the inner semicircle to the outer semicircle of the U-bend. Design B in FIG. 9B depicts the case where the U-bend had a uniform tube wall thick that is 1.25 to 5 times the straight leg tube wall thickness.

The U-bend tube wall thickness was 8.53 mm for the Baseline Design. In another embodiment, Design A, the U-bend tube wall thickness gradually changes from 21.3 mm to 8.53 mm. In another embodiment, Design B, the U-bend has a constant tube wall thickness of 14.9 mm. The process tube had a W-shaped geometry. The process tube had a total length of 14 m, a tube inside diameter of 38.1 mm, and a straight leg thickness of 8.53 mm. The U-bend diameter was 254 mm.

In the Baseline Design, Design A, and Design B, an ethane feed with a dilution steam at 670° C. was fed to the process tube with a total flow rate of 58.2 kg/hr and steam to hydrocarbon ratio of 0.3. The process outlet pressure was 72.5 kPag. An electric potential of 14.5 V was applied to the tube 5.7 meters downstream of the process inlet. The tube at both the process inlet and outlet ends were grounded. The tube material had an electric resistivity of 1.45 μOhm-m at 900° C. The thermal cracking in the process tube was represented using a literature kinetics model (Froment 1977). Flow, thermal cracking reactions, and conjugated heat transfer were modeled using Ansys Fluent v 19.4.

The predicted U-bend TMT are shown in FIG. 3B, FIG. 9A, and FIG. 9B. As seen in FIG. 3B, the Baseline Design, which had a direct electric heating coil with a U-bend wall thickness same as that of the straight legs, a nonuniform heat flux in the U-bend occurred. This is because the inner semicircle and outer semicircle of the U-bend metal wall form electric circuits that have a different length and subsequently a different electric resistance, and the resulting electric current density and joule heating power differ from the inner semicircle to the outer semicircle. Specifically, a U-bend with the same wall thickness as the straight legs of the coil led to a higher joule heating intensity at the inner semicircle of the U-bend. As seen in FIG. 3B, the temperature at the inner semicircle 280 for the Baseline Design is over 1050° C., while the outer semicircle 290 is slightly less than 950° C. These temperature differences can lead to the formations of hot spots, result in coking, and decrease product yield.

In alternative embodiments, Design A and Design B, varying U-bend geometry is depicted as part of a direct electric heating tube. Both Design A and Design B reduce the U-bend temperatures from the Baseline Case by more than 100° C. Additionally, Design A shows a relatively uniform U-bend temperature, with most of the U-bend comprising a temperature of 902° C., as depicted in FIG. 9A. Design B has some lower U-bend temperatures varying between 882° C. and 921° C., as depicted in FIG. 9B. These geometries of Designs A and B help avoid excessive TMT caused by uneven electric current density and enables the usage of U-bend of smaller semicircle, as described hereinabove.

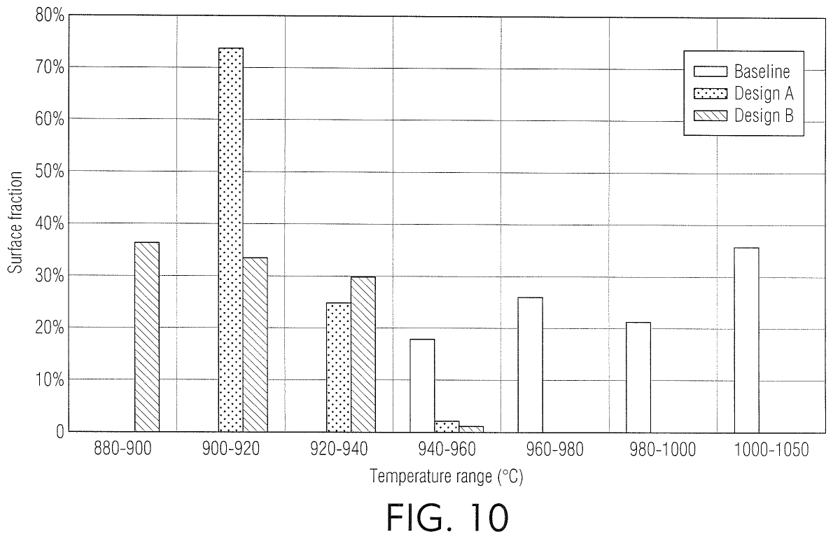

FIG. 10 is a summary graph comparing the Baseline Design and Design A and Design B. The temperatures of the surfaces of the tubes are depicted in a bar graph. As seen, the Baseline Design yields the highest temperature ranges of the surface of the tube, with temperatures exceeding 1000° C. The Baseline Design is the only design featuring temperatures beyond 960° C. Design A and Design B depict surface temperatures below 960° C. Design A's tube surface comprises temperatures mostly between 900-940° C., while Design B's tube surface comprises temperatures mostly between 880-940° C.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

Further aspects are provided by the subject matter of the following clauses

An apparatus for upgrading a hydrocarbon fluid comprising: a current voltage source; and at least one electrically conductive tube comprising: a hydrocarbon channel having a centerline; and at least one curve such that the electrically conductive tube comprises a plurality of straight portions and at least one U-shaped portion, wherein the U-shaped portion comprises: an outer wall extending between an outer semicircle of the U-shaped portion and the hydrocarbon channel and having a thickness, w_{Uo}; an inner wall extending between an inner semicircle of the U-shaped portion and the hydrocarbon channel and having a thickness, w_{Ui}, wherein a ratio of w_{Uo} to w_{Ui} is within greater than or equal to 1.2 and less than or equal to

$$2 \times {( \frac{D + d}{D - d} )}^{2} ,$$

where D is a distance between centerlines of the plurality of straight portions and d is a tube diameter, wherein the current voltage source is electrically connected to the electrically conductive tube.

The apparatus of the preceding clause, wherein the current voltage source is a DC current voltage source.

The apparatus of any preceding clause, wherein the current voltage source is an AC current voltage source.

The apparatus of any previous clause, wherein an electrical resistance along the inner semicircle of the U-shaped portion is greater than an electrical resistance of the outer semicircle of the U-shaped portion.

The apparatus of any previous clause, wherein the ratio of w_{Uo} to

$${w}_{Ui} is within \pm 30 \% of {( \frac{D + d}{D - d} )}^{2} .$$

The apparatus of any previous clause, wherein the ratio of w_{Uo} to

$${w}_{Ui} is within \pm 10 \% of {( \frac{D + d}{D - d} )}^{2} .$$

The apparatus of any previous clause, wherein w_{Uo} gradually increases from the plurality of straight portions to the U-shaped portion.

The apparatus of any previous clause, wherein the plurality of straight portions of the electrically conductive tube has a uniform tube wall thickness (w); w_{U} is the wall thickness of the U-shaped portion, where w_{Uo} is equal to w_{Ui}; and where w_{U} is greater than or equal to 1.25w and less than or equal to 5.00w.

The apparatus of the previous clause, wherein w_{U} is greater than or equal to 2.00w and less than or equal to 4.00w.

The apparatus of any previous clause, wherein the at least one electrically conductive tube comprises a first electrically conductive tube and a second electrically conductive tube, wherein the first electrically conductive tube is electrically connected to the second electrically conductive tube.

The apparatus of the previous clause, wherein the first electrically conductive tube and the second electrically conductive tube are configured such that current generated from the current voltage source flows from the first electrically conductive tube to the second electrically conductive tube and from the second electrically conductive tube to the current voltage source.

The apparatus of any of the previous two clauses, wherein the apparatus further comprises a positive conductor and a negative conductor.

The apparatus of the previous clause, wherein the positive conductor electrically connects the current voltage source and the first electrically conductive tube and the negative conductor electrically connects the current voltage source and the second electrically conductive tube.

The apparatus of the previous four clauses, wherein the apparatus is configured to: introduce a hydrocarbon fluid into an inlet of the first electrically conductive tube; apply current to the first electrically conductive tube to heat the hydrocarbon fluid within the first electrically conductive tube to reaction temperature to form a first product stream; pass the first product stream from an outlet of the first electrically conductive tube to an inlet of the second electrically conductive tube; and apply current to the second electrically conductive tube to heat the first product stream within the second electrically conductive tube to reaction temperature to form a second product stream.

An apparatus for upgrading a hydrocarbon fluid comprising: a current voltage source; and at least one electrically conductive tube comprising: a hydrocarbon channel having a centerline; at least one curve such that the electrically conductive tube comprises a plurality of straight portions and at least one U-shaped portion, wherein the current voltage source is electrically connected to the electrically conductive tube and wherein the plurality of straight portions comprises a first straight portion connected to a first end of the U-shaped portion and a second straight portion connected to a second end of the U-shaped portion such that the first straight portion and the second straight portion are separated by the U-shaped portion; an electrical coupler electrically connecting the first straight portion and the second straight portion, and a resistance of the electrical coupler is less than half a resistance of the U-shaped portion.

The apparatus of the previous clause, wherein the at least one electrical coupler comprises a flexible, metallic, electrically conducting cable.

The apparatus of any previous clause, wherein the at least one electrical coupler comprises at least one of a nickel-rich alloy, a chrome-rich alloy, or aluminum alloy.

The apparatus of any previous clause, wherein the at least one electrical coupler is flexible.

The apparatus of any previous clause, wherein the at least one electrical coupler is rigid.

The apparatus of any previous clause, wherein the at least one electrical coupler is inside the U-shaped portion.

The apparatus of any previous clause, wherein the at least one electrical coupler is outside the U-shaped portion.

What is claimed is:

1. An apparatus for upgrading a hydrocarbon fluid comprising:
at least one electrically conductive tube comprising a hydrocarbon channel having a centerline and at least one curve, such that the electrically conductive tube comprises a plurality of straight portions and at least one U-shaped portion, wherein the U-shaped portion comprises:
an outer wall extending between an outer semicircle of the U-shaped portion and the hydrocarbon channel and having a thickness, w_{Uo}, and
an inner wall extending between an inner semicircle of the U-shaped portion and the hydrocarbon channel and having a thickness, w_{Ui}, wherein a ratio of w_{Uo} to w_{Ui} is within greater than or equal to 1.2 and less than or equal to
$$2 \times {( \frac{D + d}{D - d} )}^{2} ,$$
where D is a distance between centerlines of the plurality of straight portions and d is a tube diameter; and
a current voltage source electrically connected to the electrically conductive tube.

2. The apparatus of claim 1, wherein the current voltage source is a DC current voltage source.

3. The apparatus of claim 1, wherein the current voltage source is an AC current voltage source.

4. The apparatus of claim 1, wherein an electrical resistance along the inner semicircle of the U-shaped portion is greater than an electrical resistance of the outer semicircle of the U-shaped portion.

5. The apparatus of claim 1, wherein the ratio of w_{Uo} to
$${w}_{Ui} is within \pm 30 \% of {( \frac{D + d}{D - d} )}^{2} .$$

6. The apparatus of claim 1, wherein the ratio of w_{Uo} to
$${w}_{Ui} is within \pm 10 \% of {( \frac{D + d}{D - d} )}^{2} .$$

7. The apparatus of claim 1, wherein w_{Uo} gradually increases from the plurality of straight portions to the U-shaped portion.

8. The apparatus of claim 1, wherein
the plurality of straight portions of the electrically conductive tube has a uniform tube wall thickness (w);
w_{U} is the wall thickness of the U-shaped portion, where w_{Uo} is equal to w_{Ui}; and
where w_{U} is greater than or equal to 1.25w and less than or equal to 5.00w.

9. The apparatus of claim 8, wherein w_{U} is greater than or equal to 2.00w and less than or equal to 4.00w.

10. The apparatus of claim 1, wherein the at least one electrically conductive tube comprises a first electrically conductive tube and a second electrically conductive tube, wherein the first electrically conductive tube is electrically connected to the second electrically conductive tube.

11. The apparatus of claim 10, wherein the first electrically conductive tube and the second electrically conductive tube are configured such that current generated from the current voltage source flows from the first electrically conductive tube to the second electrically conductive tube and from the second electrically conductive tube to the current voltage source.

12. The apparatus of claim 10, wherein the apparatus further comprises a positive conductor and a negative conductor.

13. The apparatus of claim 12, wherein the positive conductor electrically connects the current voltage source and the first electrically conductive tube and the negative conductor electrically connects the current voltage source and the second electrically conductive tube.

14. The apparatus of claim 10, wherein the apparatus is configured to:
introduce a hydrocarbon fluid into an inlet of the first electrically conductive tube;
apply current to the first electrically conductive tube to heat the hydrocarbon fluid within the first electrically conductive tube to reaction temperature to form a first product stream;
pass the first product stream from an outlet of the first electrically conductive tube to an inlet of the second electrically conductive tube; and
apply current to the second electrically conductive tube to heat the first product stream within the second electrically conductive tube to reaction temperature to form a second product stream.

15. An apparatus for upgrading a hydrocarbon fluid comprising:
a current voltage source; and
at least one electrically conductive tube comprising a hydrocarbon channel having a centerline and at least one curve, such that the electrically conductive tube comprises a plurality of straight portions and at least one U-shaped portion, wherein:
the current voltage source is electrically connected to the electrically conductive tube, and
the plurality of straight portions comprise a first straight portion connected to a first end of the U-shaped portion and a second straight portion connected to a second end of the U-shaped portion, such that the first straight portion and the second straight portion are separated by the U-shaped portion; and
at least one electrical coupler electrically connecting the first straight portion and the second straight portion, wherein a resistance of the electrical coupler is less than half a resistance of the U-shaped portion.
